(19) United States
(12) Patent Application Publication
(10) Pub. No.: US 2020/0256937 A1
(43) Pub. Date: Aug. 13, 2020

(54) Radio-Frequency Coil Device and Magnetic Resonance Imaging Apparatus Having the Same

(71) Applicant: Hitachi, Ltd., Tokyo (JP)

(72) Inventors: Kohjiro IWASAWA, Tokyo (JP); Kazuyuki KATO, Tokyo (JP); Hideta HABARA, Tokyo (JP); Yosuke OTAKE, Tokyo (JP); Satoshi YAMASHITA, Tokyo (JP)

(21) Appl. No.: 16/778,086

(22) Filed: Jan. 31, 2020

(30) Foreign Application Priority Data
Feb. 13, 2019 (JP) 2019-023246

(51) Int. Cl. G01R 33/34; A61B 5/00; A61B 5/055

(52) U.S. Cl. CPC G01R 33/34084; G01R 33/34007; A61B 2562/164; A61B 5/055; A61B 5/0042

(57) ABSTRACT

An object of the present invention is to provide a radio-frequency coil device that can be brought into close contact with a subject regardless of a size of the subject and can obtain a high-quality MRI image. The radio-frequency coil device includes at least one flexible coil unit, and a fixture is disposed to surround an outer periphery of the coil unit. The fixture includes, for example, a pair of panels disposed on both sides of the coil unit, and a fixing belt that is provided between the panels and fastens and fixes the panels. The coil unit is deformed by fastening the fixture, to be disposed in close contact with various head shapes.

The present application claims priority from Japanese patent application JP-2019-23246 filed on Feb. 13, 2019, the content of which is hereby incorporated by reference into this application.

### BACKGROUND OF THE INVENTION

### Field of the Invention

The present invention relates to a magnetic resonance imaging (MRI) apparatus, and particularly, to an RF coil (Radio Frequency Coil: high-frequency coil) that irradiates a high-frequency magnetic field and detects a nuclear magnetic resonance signal.

### Background Art

The MRI apparatus is a medical image diagnostic apparatus that causes nuclear magnetic resonance to occur in a nuclear spin in any region of an inspection object, to obtain an image of the region from a generated nuclear magnetic resonance signal.

In MRI imaging, a high-frequency magnetic field is irradiated by the high-frequency coil while applying a gradient magnetic field to a subject placed in a static magnetic field. When the nuclear spin in the subject, for example, the nuclear spin of a hydrogen atom is excited by irradiation of the high-frequency magnetic field, and the excited nuclear spin returns to an equilibrium state, a circularly polarized magnetic field is generated as the nuclear magnetic resonance signal. This signal is detected by the high-frequency coil and subjected to signal processing, so that a hydrogen nuclei distribution in a living body is imaged.

For example, in the case of a receiver coil that detects the nuclear magnetic resonance signal, a surface coil that can be disposed in the vicinity of the subject is generally used as the high-frequency coil. This is because as a distance between the high-frequency coil and the subject is smaller, a signal acquisition efficiency is higher, and an MRI image quality can be higher. Usually, as the surface coil, the receiver coil for each application based on a shape of an imaging object such as head, chest, or extremities is used. The receiver coil for the head that is generally commercialized has a coil housing having a fixed size placing importance to durability. In order to address different sizes for each subject and changes in body posture during imaging, the receiver coil for the head having a mechanism capable of changing the size has also been proposed (for example, see

### SUMMARY OF THE INVENTION

A general receiver coil for head with a fixed size is designed with a size suitable for a large subject in order to increase population coverage. Therefore, for many subjects, there is a problem that the distance between the coil and the subject is large and the signal acquisition efficiency is reduced. Further, in order to prevent reduction in the MRI image quality due to body movement, a fixing operation is performed in which a sponge is filled in a space between the coil housing and the subject. In this operation, a technician changes the number and shape of sponges based on a size of the head so that sufficient fixation can be obtained. However, since the operation of filling the sponge needs to be performed in a narrow space between the coil housing and the subject, there is a problem that the operation takes time.

On the other hand, the coil device described in JP-A-2015-000098 includes a unit for moving up and down a first head coil disposed above the head of the subject placed on a headrest with respect to a second head coil disposed on the headrest, with the head of the subject who is laid down in a lateral position being placed on the headrest. The coil device can change a coil size to fit the size of the subject by changing a distance between the housings of the two head coils.

However, since the coil device has a structure in which the head is sandwiched from left and right by a rigid coil housing, a gap is generated between the two housings when the coil size is increased, and there is a problem that sensitivity of the coil in the gap falls and the MRI image quality is deteriorated. Further, there is a problem that the body movement cannot be sufficiently suppressed in a direction in which the subject nods just by sandwiching the head from the left and right sides of the head.

The present invention has been made in view of the above circumstances, and objects of the present invention are to provide a radio-frequency coil device that can be brought into close contact with the subject regardless of the size of the subject, and to provide a high-quality MRI image by using the radio-frequency coil device.

The present invention includes at least one flexible coil unit. A fixture is disposed to surround an outer periphery of the coil unit. The fixture includes, for example, a pair of panels disposed on both sides of the coil unit, and a fixing belt that is provided between the panels and fastens and fixes the panels. The coil unit can be disposed in close contact with various head shapes by deformation by fastening the fixture.

That is, the radio-frequency coil device of the present invention is a radio-frequency coil device for a magnetic resonance imaging apparatus, including a flexible coil unit, and a fixture disposed outside the coil unit with respect to an inspection object. The fixture includes a fastener that brings the coil unit attached to the inspection object into close contact with the inspection object.

Further, the MRI apparatus of the present invention includes the radio-frequency coil device of the present invention as a transmitter coil or a receiver coil.

According to the present invention, it is possible to shorten the fixing operation for bringing the surface coil into close contact with the subject. The surface coil can be in close contact with the subject regardless of the size of the subject, and the high-quality MRI image can be provided.

### BRIEF DESCRIPTION OF THE DRAWINGS

FIG. 1 is a block diagram showing an overall configuration of an MRI apparatus to which the present invention is applied;

FIG. 2 is a diagram showing a relationship between an RF transmitter coil and an RF receiver coil in the MRI apparatus;

FIGS. 3A and 3B are diagrams showing an outline of a radio-frequency coil device, FIG. 3A is a diagram viewed from above a subject, and FIG. 3B is a diagram of an XZ plane;

FIGS. 4A and 4B are diagrams showing an example in which a coil unit is an array coil, FIG. 4A is a development diagram showing a coil body, and FIG. 4B is a diagram showing a cross-section of the coil body including a housing;

FIG. 5 is a perspective view of the radio-frequency coil device of a first embodiment;

FIG. 6A is a diagram showing an arrangement example of the coil body when the radio-frequency coil device of the first embodiment is applied to a head coil, and FIG. 6B is a diagram of the head coil viewed from the side;

FIGS. 7A and 7B are diagrams showing an attaching example of the radio-frequency coil device of the first embodiment, FIG. 7A is a diagram showing a close contact with a relatively large inspection object, and FIG. 7B is a diagram showing a close contact with a relatively small inspection object;

FIGS. 8A to 8C are diagrams showing positions of the radio-frequency coil device of the first embodiment, FIG. 8A shows a position during head inspection, FIG. 8B shows a retracted position, and FIG. 8C shows an arrangement to the face side;

FIG. 9A is a circuit diagram of a sub-coil of the radio-frequency coil device of the first embodiment, and FIGS. 9B and 9C are diagrams showing examples of a transmission-reception magnetic coupling prevention circuit inserted into the sub-coil;

FIG. 10 is a perspective view showing Modification 1 of the radio-frequency coil device of the first embodiment;

FIGS. 11A and 11B are diagrams showing Modification 2 of the radio-frequency coil device of the first embodiment; and

FIG. 12A is a circuit diagram of a sub-coil of the radio-frequency coil device of the second embodiment, and FIG. 12B is a diagram showing an example of the transmission-reception magnetic coupling prevention circuit inserted into the sub-coil.

### DESCRIPTION OF THE PREFERRED EMBODIMENTS

First, an embodiment of an MRI apparatus to which a radio-frequency coil of the present invention is applied will be described.

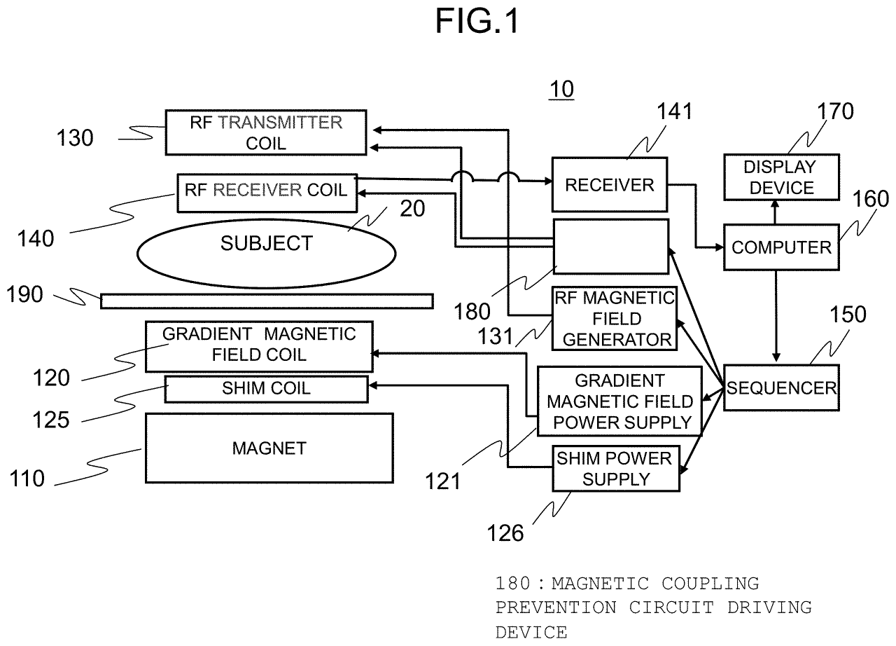

FIG. 1 is a block diagram showing a schematic configuration of an MRI apparatus 10. As shown in the drawing, the MRI apparatus 10 includes, as main components, a magnet 110 that generates a static magnetic field, a gradient magnetic field coil 120 that gives a magnetic field gradient to the static magnetic field, a shim coil 125 that adjusts uniformity of the static magnetic field, an RF transmitter coil 130 that generates a high-frequency magnetic field, an RF receiver coil 140 that receives a nuclear magnetic resonance signal, a sequencer 150, a computer (PC) 160 that performs calculations such as image reconstruction and controls each part of the MRI apparatus, and a display device 170 that displays an image, a GUI or the like.

The magnet 110 may be a horizontal magnetic field type and a vertical magnetic field type depending on a direction of the generated static magnetic field, and the present invention can be applied to either type. Hereinafter, the horizontal magnetic field type will be described as an example. In the drawing, the direction of the static magnetic field is shown as a Z direction (hereinafter the same). An inspection object (generally human: hereinafter also referred to as a subject) 20 is placed in a space of the static magnetic field generated by the magnet 110 while being placed on a table 190.

The gradient magnetic field coil 120 is connected to a gradient magnetic field power supply 121 and generates a gradient magnetic field. The shim coil 125 is connected to a shim power supply 126 and adjusts the uniformity of the magnetic field. The RF transmitter coil 130 is connected to a high-frequency magnetic field generator 131 and irradiates (transmits) the high-frequency magnetic field to the inspection object 20. The RF receiver coil 140 is connected to a receiver 141 and receives the nuclear magnetic resonance signal from the inspection object 20. The RF transmitter coil 130 and the RF receiver coil 140 are connected with a magnetic coupling prevention circuit (not shown) for preventing mutual magnetic coupling, and the magnetic coupling prevention circuit is driven by a magnetic coupling prevention circuit driving device 180.

The sequencer 150 sends instructions to the gradient magnetic field power supply 121, the high-frequency magnetic field generator 131, and the magnetic coupling prevention circuit driving device 180 according to instructions from the computer (PC) 160 to operate them, and sets a magnetic resonance frequency as a reference for demodulation in the receiver 141.

The computer (PC) 160 controls an operation of the entire MRI apparatus 10 and performs various signal processing. For example, according to the instruction from the sequencer 150, the high-frequency magnetic field is applied to the inspection object 20 through the RF transmitter coil 130. The nuclear magnetic resonance signal generated from the inspection object 20 by irradiating the high-frequency magnetic field is detected by the RF receiver coil 140 and demodulated by the receiver 141. The signal demodulated by the receiver 141 is received through an A/D conversion circuit, and signal processing such as image reconstruction is performed. Its result is displayed on the display device 170.

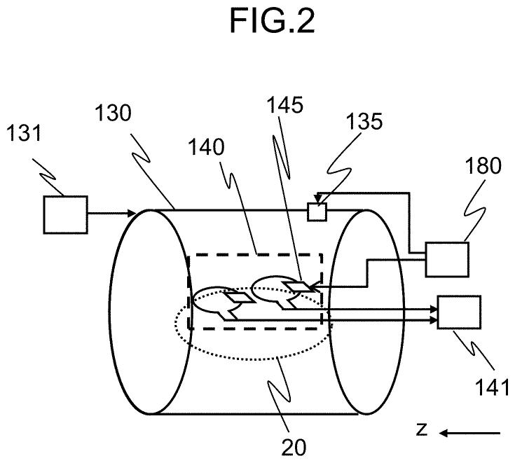

Next, an arrangement of the RF transmitter coil 130 and the RF receiver coil 140 will be described with reference to FIG. 2. FIG. 2 shows an example in which a general birdcage type RF coil (whole body coil) is used as the RF transmitter coil 130 and an array coil with surface coils arranged side by side is used as the RF receiver coil 140. Although only two surface coils (referred to as sub-coils) constituting the array coil are shown in the drawing, the number of sub-coils is arbitrary.

The birdcage type RF coil used as the RF transmitter coil 130 is disposed inside (inspection space side) the magnet 110 of a cylindrical horizontal magnetic field method and has a shape in which two ring-shaped conductors are connected by a plurality of linear conductors. A capacitor adjusting element (not shown) is inserted into one or both of the ring-shaped conductor and the linear conductor, and the resonance frequency is adjusted to a resonance frequency of an element to be excited. Further, a magnetic coupling prevention circuit 135 is inserted in series at at least one location of the conductor. The magnetic coupling prevention circuit 135 includes, for example, a PIN diode, and controls on/off of the PIN diode by a control signal (DC current) from the magnetic coupling prevention circuit driving device 180. That is, during high-frequency signal transmission, a control current for turning on the PIN diode is supplied, so that the birdcage type RF coil is caused to function as the RF transmitter coil 130. When receiving the nuclear magnetic resonance signal, the control current is stopped, so that impedance of the birdcage type RF coil is increased, and the PIN diode is turned off.

The array coil (RF receiver coil 140) is arranged so that each sub-coil is as close as possible to the inspection object 20, and has a structure that can be attached to and detached from the inspection object 20.

In each sub-coil, the adjusting element such as a capacitor is inserted into the conductor constituting the coil, and its resonance frequency is adjusted to be a resonance frequency at which the nuclear magnetic resonance signal of the element that can be excited by the birdcage type RF coil 130 can be detected. Further, similarly to the RF transmitter coil 130, a magnetic coupling prevention circuit 145 is connected to each sub-coil. The magnetic coupling prevention circuit 145 includes, for example, a parallel resonance circuit that has a high impedance at a frequency of the nuclear magnetic resonance signal, and drives the magnetic coupling prevention circuit 145 by a direct current (control current) from the magnetic coupling prevention circuit driving device 180 during high-frequency signal transmission, so that the parallel resonance circuit included in the magnetic coupling prevention circuit 145 has a high impedance and magnetic coupling with the RF transmitter coil 130 is eliminated.

FIG. 2 shows an example in which the array coil is used as the RF receiver coil 140, but it is also possible to use such an array coil as the RF transmitter coil 130.

The MRI apparatus according to the present embodiment uses a radio-frequency coil device with improved attaching characteristics to the inspection object as the RF transmitter coil 130 or the RF receiver coil 140. Hereinafter, embodiments of the radio-frequency coil device will be described. In the following embodiments, embodiments applied to a head coil will be described, however, the radio-frequency coil device of the present invention is not limited to the head coil.

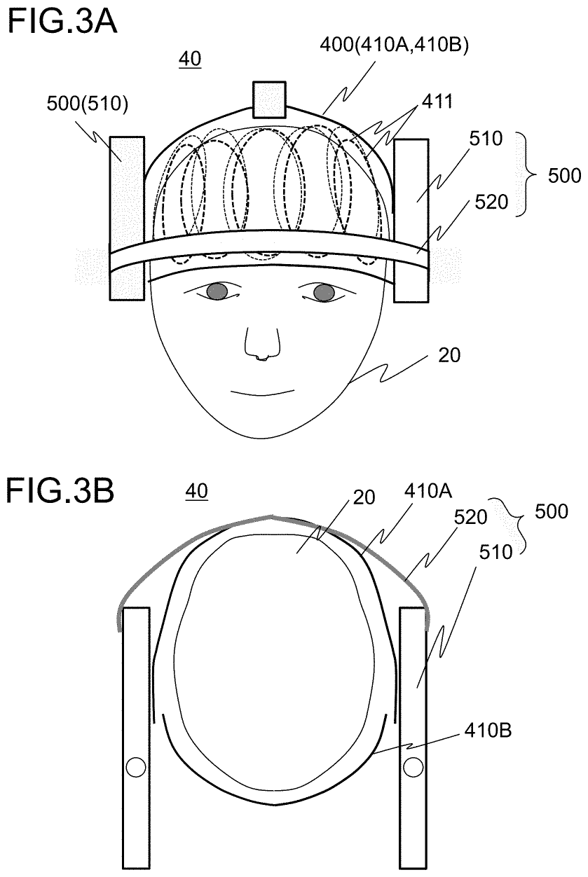

First, an outline of a radio-frequency coil device 40 common to each embodiment described below will be described with reference to FIG. 3.

As shown in FIGS. 3A and 3B, the radio-frequency coil device 40 includes a coil body 400 including one or more coil units, and a fixture 500 for fixing the coil body 400 to the inspection object (subject) 20.

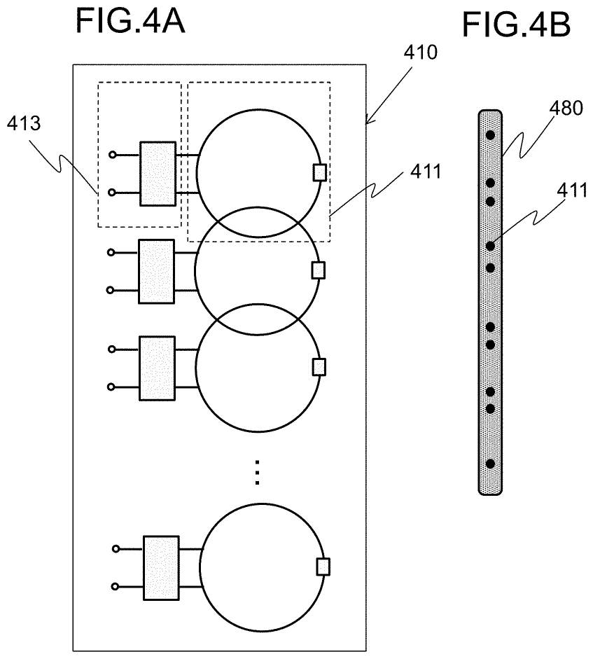

In an example shown in FIG. 3, the coil body 400 includes two coil units 410A and 410B. When the two coil units are not to be distinguished, they are denoted by a reference numeral 410. As shown in FIG. 4A, each coil unit 410 includes one or more (a plurality in the drawing) loop coil portions 411, and a circuit unit (circuit substrate) 413 that connects the loop coil portion 411 and an external cable (not shown). As shown in FIG. 4B, at least the loop coil portion 411 is covered with a flexible housing 480. The housing 480 is made of a sponge, an elastomer, a flexible material such as net or fiber, and its shape can be arbitrarily changed. For example, FIG. 4B shows a straight cross-section, however, it can be deformed into a curved shape along a surface of the subject.

Along with deformation of the housing 480, a distance and a degree of overlap between (the loop coil portions of) the two coil units 410 also change. In addition, when there are plural loop coil portions 411, the distance and the degree of overlap between the loop coil portions 411 also change. The coil unit 410 is adjusted so that the mutual magnetic coupling is minimized even when there is such a change in position between the loop coil portions. Details of adjustment of the coil will be described below.

The loop coil portion 411 in each coil unit 410 is disposed so that its loop plane has an angle with respect to an X-Y plane in order to detect a magnetic field (rotating magnetic field) in a direction perpendicular to the direction (Z direction in the drawing) of the static magnetic field of the MRI apparatus. The loop surface is most preferably disposed to be substantially parallel to the Z direction.

The fixture 500 is disposed outside the coil body 400 with respect to the subject 20 and has a structure that can bring the coil body 400 into close contact with the subject 20 from the left, right, and upper sides of the subject. Further, it is also possible to adopt a structure that can bring the coil body 400 into close contact with the subject 20 from a parietal side.

As described above, the radio-frequency coil device of the present embodiment has the flexible coil body 400 and includes the structure (fixture 500) that can bring the coil body 400 into close contact with the subject outside the coil body 400, so that it can be attached to the subject with good workability and it is possible to reliably bring the coil body and the subject into close contact with each other. Therefore, an MR image quality can be improved whether the radio-frequency coil device is used as the transmitter coil or as the receiver coil.

Next, detailed embodiments of the radio-frequency coil device will be described.

### First Embodiment

The radio-frequency coil device 40 of the present embodiment includes the flexible coil body 400 and the fixture 500 that brings the coil body 400 into close contact with the subject 20. The coil body 400 includes the two coil units, and each coil unit is the array coil including a plurality of sub-coils. The fixture 500 includes a pair of panels 510 and a fixing belt 520 (fastener) that fastens the pair of panels, and further includes a support unit 530 that supports the coil body.

The sub-coils constituting the coil unit (array coil) are adjusted to make parallel resonance (resonate in parallel) at the frequency of the nuclear magnetic resonance signal, and are adjusted to prevent the magnetic coupling between the sub-coils even when a distance between the sub-coils varies due to deformation of the coil body.

### [Features of Structure]

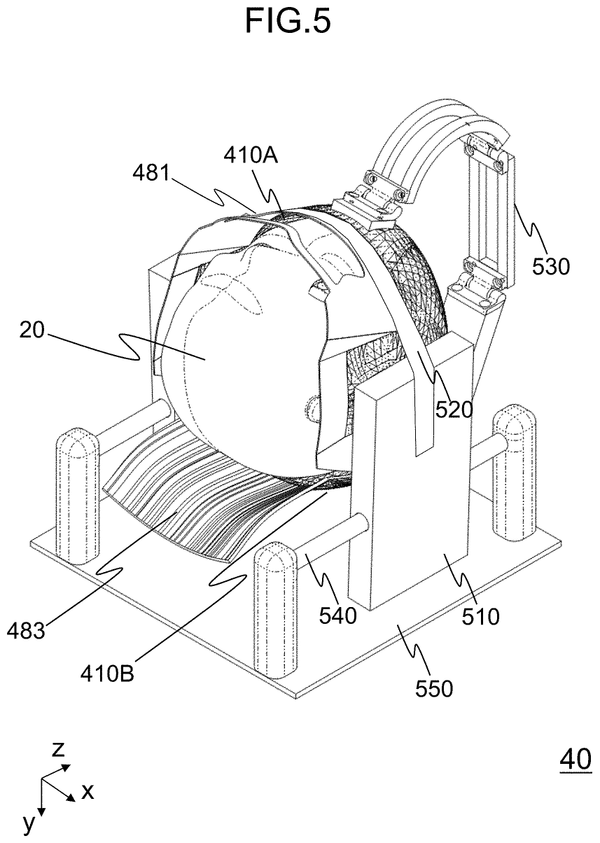

Hereinafter, a structure of the radio-frequency coil device of the present embodiment will be described in detail with reference to FIGS. 5 to 8.

As shown in FIG. 5, the coil body 400 has the coil unit 410A including the array coil in which the loop coil portion is disposed to cover at least apart of a temporal region from a frontal region of the subject, and the coil unit 410B including the array coil in which the loop coil portion is disposed to cover at least a part of the temporal region from an occipital region. The coil unit 410A is fixed to the support unit 530, and the coil unit 410B is fixed to the fixture 500.

Each coil unit 410 is incorporated in the housing (FIG. 4B) 480 made of a flexible member such as the sponge or resin, and has a feature of retaining its outer shape when an external force is not applied, and capable of deforming when the external force is applied. Thus, when the coil body 400 is brought close to the subject 20 with the fixture 500 (panels 510) described below, it can be deformed and brought into close contact with the subject 20.

Further, the housing 480 may be provided with an attachment for improving feeling of wearing for the subject. In an example shown in FIG. 5, a viewing window 481 for securing a field of view of the subject, a protective sheet 483 for protecting a neck of the subject, and the like are provided as such attachments.

As shown in FIG. 4, the coil unit 410 (array coil) has a plurality of sub-coils 420 arranged, each including the loop coil portion 411 and a power supply substrate 413 including, for example, a low impedance circuit 430 for connecting the loop coil portion 411 to the external cable. Each loop coil portion 411 is inserted with the transmission-reception magnetic coupling prevention circuit 145 and a circuit element such as the capacitor for adjustment. Electrical characteristics of the sub-coil 420 and adjustment by the adjusting circuit element will be described in detail below.

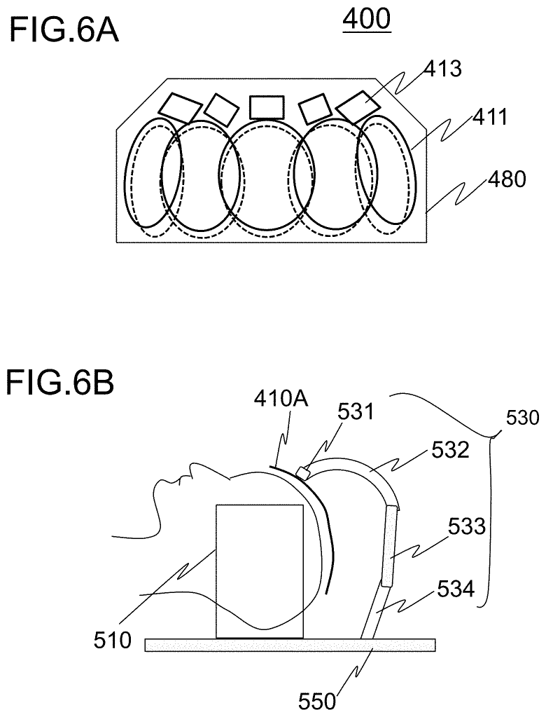

When the radio-frequency coil device is the head coil, the power supply substrate 413 of the sub-coil in the coil unit 410 maybe concentrated on the parietal side as shown in FIG. 6A. Thus, it is possible to suppress physical collision between the power supply substrate 413 and the fixing panels 510 when the coil is attached by the fixture 500.

The fixture 500 includes the pair of panels 510, the fixing belt 520, and a base 550 that fixes the panels 510 and the support unit 530. The fixing belt 520 is fixed at a position close to upper ends of the pair of panels 510 and can be fastened by a fastening unit such as a hook-and-loop fastener and a buckle. A shaft 540 is fixed to be parallel to a surface of the base 550, and the panel 510 is fixed to be rotatable about the shaft 540. Further, the panel 510 may be movable in the Z direction with the shaft 540 as a guide. In such a structure, the panel 510 can be rotated about the shaft 540 to increase or decrease a gap between the upper ends of the pair of panels 510, and the coil unit 410 present between the panel 510 and the subject 20 can be brought into close contact with the subject 20 by tightening the fixing belt 520. When the panel 510 is movable along the axis, a position of the panel 510 in the Z direction can also be adjusted.

As shown in FIG. 6B, the support unit 530 is a mechanism for bringing the coil body 400 (coil unit 410A) into close contact with the subject on the parietal side. In the shown embodiment, the support unit 530 includes a fixing portion 531 fixed to coil unit 410A, a fixing portion fixed to the base 550 (not shown), and a plurality of arms 532 to 534 that are flexibly connected to the fixing portions and flexibly connected to each other. In the shown example, the arm 532 fixed to the fixing portion 531 has an arch shape, and is configured to ensure a high degree of freedom in attachment position of the coil body.

The support unit 530 can arbitrarily change a position of the coil unit 4 1 0A within a predetermined movable range by changing an angle of a hinge portion connecting the arms, thereby bringing the coil unit 410A into close contact with the head.

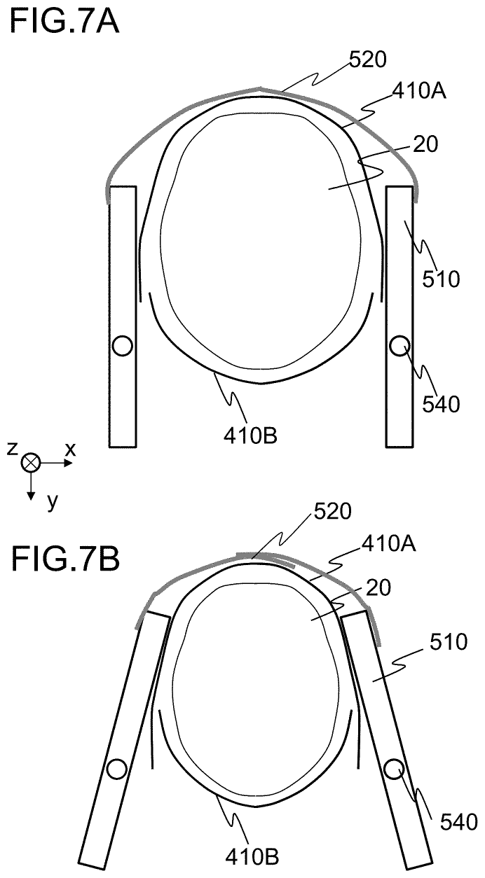

Next, functions of the radio-frequency coil device of the present embodiment will be described. First, the function that allows the radio-frequency coil device to be closely disposed with respect to various subject sizes by the fixture 500 will be described with reference to FIGS. 7A and 7B.

FIG. 7A shows a cross-sectional view when the coil body (array coil) 400 is disposed on the head of the relatively large subject 20. This cross-section is a cross-section perpendicular to a body axis direction near eyebrows of the head. As shown in FIG. 7A, the panels 510 are brought close to a face by tightening the fixing belt 520, and the flexible coil units 410A and 410B can be arranged in close contact with a head shape by deformation. Thus, a signal acquisition efficiency is improved and the image quality is improved.

FIG. 7B shows a cross-sectional view when the coil body (array coil) 400 is disposed on the head of the small subject 20. This cross-section is a cross-section perpendicular to the body axis direction near the eyebrows of the head similarly to FIG. 7A. By operating the support unit 530 (not shown in FIG. 7) to reduce a distance between the coil unit 410A on the face side and the coil unit 410B on the occipital side, the flexible coil units 410A and 410B can also be arranged in close contact with a small head shape. By tightening the fixing belt 520, the fixing panels 510 are brought close to the face, and the flexible array coil units 410A and 410B can be arranged in close contact with the head shape by deformation. Thus, the signal acquisition efficiency is improved and the image quality is improved.

As described above, since the flexible coil units 410A and 410B can cover the head without the gap regardless of the size of the head of the subject, it is possible to prevent sensitivity of the coil from decreasing due to the gap. Further, body movement can be prevented by the fixture 500. Specifically, the body movement such as rotation of shaking head side to side or cocking rotation of the subject is suppressed by the fixing panels 510. Further, since a forehead of the subject is pressed by the fixing belt 520, the body movement in a nodding direction of the subject can be suppressed as compared with a case where the head is only sandwiched from the left and right sides.

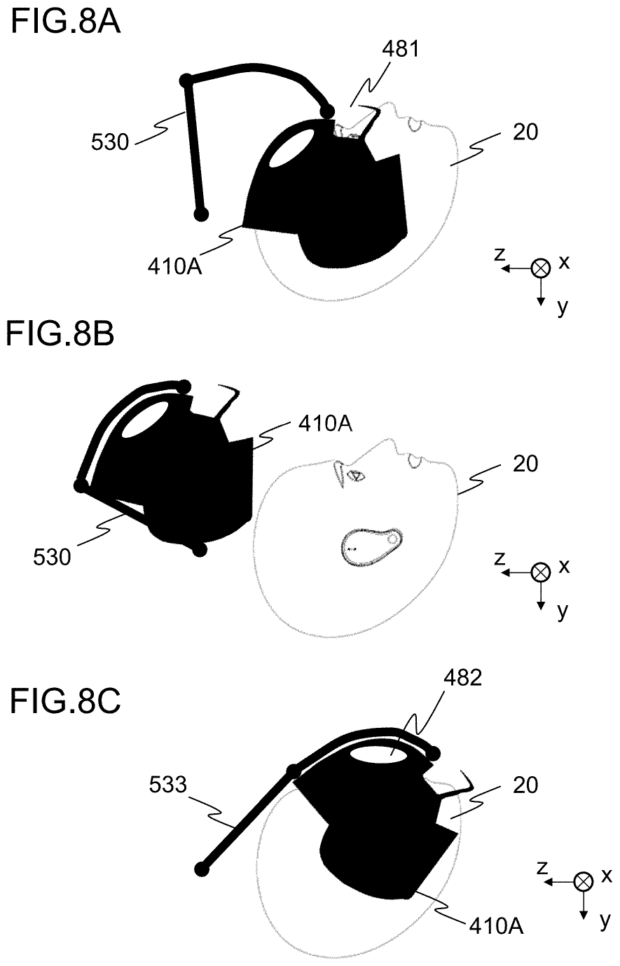

Next, the function of the support unit 530 will be described. FIGS. 8A to 8C show states where the coil unit 410A is attached by the arms 532 to 534. In FIGS. 8A to 8C, connecting portions of the arm are shown by black circles, and the coil unit 410A is shown blackened. The arm 534 and the fixing portion 531 are not shown.

FIG. 8A is a diagram showing a state when a brain of the subject 20 is examined. In this state, the coil unit 410A supported by the support unit 530 is provided to cover a frontal lobe side of the brain of the subject 20. FIG. 8B is a diagram showing a state when the coil unit 410A is retracted to the parietal side of the subject 20. In this state, it is possible to prevent the coil unit 410A from interfering with the subject 20 when the subject 20 lies on a bed or gets up from the bed. FIG. 8C is a diagram showing a state when an oral cavity region of the subject 20 is examined.

An operation of providing and fixing the radio-frequency coil device 40 having such a structure on the subject is performed as follows. First, as shown in FIG. 8B, with the coil unit 410A retracted, the subject is laid in a supine position on the coil unit 410B on the occipital side. Then, the coil unit 410A is provided as shown in FIG. 8A. The fixing belt 520 is tightened and fixed with the hook-and-loop fastener or the like. Thus, a providing and fixing operation of the coil body 400 of the present embodiment on the subject is generally completed. This reduces time of the fixing operation of filling the sponge which has been generally performed so far. In general, the fixing operation has been performed in a narrow space between the coil housing and the subject. In contrast, in the present embodiment, since the fixing belt 520 can be operated using a wide space outside the coil unit 410A, a physical burden due to a posture of an engineer can be reduced.

In an example shown in FIGS. 8A to 8C, the coil unit 410A includes viewing windows 481 and 482 corresponding to two positions (FIGS. 8A and 8C) as the attachment for improving the feeling of wearing of the subject. That is, the viewing window 481 is a window or opening made of a light-transmitting member formed in a housing portion that bulges from the loop coil portion of the coil unit 410A. At the attachment position shown in FIG. 8A, the viewing window 481 is located in front of eyes of the subject. The viewing window 482 is formed in each portion where there is no conductor (a portion surrounded by a loop) of the two loop coil portions. The viewing window 482 is a window or opening made of the light-transmitting member similar to the viewing window 481, and is located in front of the eyes of the subject at the attachment position shown in FIG. 8C.

Thus, the field of view of the subject 20 is secured by the first viewing window 481 in a state of FIG. 8A and by the second viewing window 482 in a state of FIG. 8C.

The support unit 530 may be provided with a limiting unit that limits a movable range of the arm. The movable range may be limited by, for example, providing a stopper on the hinge so that the arm of the support portion 530 does not protrude beyond a bed length to the parietal side during retraction. Thus, when the bed is moved up and down, the support unit 530 can be prevented from physically colliding with a gantry. A mechanism constituting the limiting unit is not particularly limited, and a manually operated stopper may be provided, or a mechanism for limiting the movable range of the arm may be provided at the connecting portion of the arm.

Further, although a case where the support unit 530 is configured by a combination of the rigid arm and the hinge has been described, it is not limited thereto as long as the support unit 530 is a mechanism that moves the coil body 400 from the retracted position to the attachment position. For example, it may be configured with a ball joint. Thus, a degree of freedom of a movable direction increases.

According to the present embodiment, the coil body 400 attached to the subject has a flexible structure, and the fixture 500 (the panel 510 and the fixing belt 520) that brings the coil body 400 into close contact with the subject from at least three directions outside the coil body 400 is provided. Thus, it is possible to arrange an entire coil close to the subject with a simple operation and to prevent the body movement of the subject.

Further, according to the present embodiment, by providing the support unit 530 having a wide movable range in addition to the fixture 500, the coil body 400 can be brought into close contact with the subject also from the parietal side. Further, since the movable range of the support unit 530 is from a position where the coil unit 410 on the face side retracts to the parietal side of the coil unit 410 on the occipital side to a position where a part of the coil unit 410 on the face side reaches a chin of the subject, an examination of the brain and an examination of a face portion can be performed together without requiring an additional array coil. Furthermore, by providing the two viewing windows 481 and 482 corresponding to the movable range, it is possible to give openness to the face portion and reduce a feeling of pressure felt by the subject.

The structure and a mechanical function of the radio-frequency coil device 40 of the present embodiment have been described above. The structure of the radio-frequency coil device 40 of the present invention, however, is not limited to the above description.

For example, although a case where there are two coil units constituting the coil body 400 has been shown, the number of coil units is not limited to two and may be one or three or more. However, when there are two coil units, it is possible to arrange them in close contact with various subject sizes as compared with a case where only one coil unit is used. And there is an effect that a time of providing and fixing operation can be reduced as compared with a case where there are many units. Further, when there are, for example, three or more coil units, the subject size can be widely covered even if a variable amount overlapping between different coil units is reduced. If the variable amount overlapping between different coil units is reduced, the magnetic coupling between the sub-coils can be reduced, and reduction of the MRI image quality can be prevented.

Further, in the present embodiment, although an example in which the coil unit 410 is disposed on the head of the subject 20 has been shown, an object part is not limited to the head. By devising a shape of the coil unit 410, the same effect can be obtained, for example, even if an abdomen or extremities are the object part. Further, in the present embodiment, although an example in which the two coil units 410 are provided on the head of the subject has been shown, it is not limited to this. For example, the image may be taken only with the coil unit 410B without using the coil unit 410A. Thus, it is possible to easily image only the occipital region or even a smaller inspection object such as a neonatal head.

### [Adjustment of Coil Unit]

Next, the adjustment of the electrical characteristics of the coil unit 410 arranged close to the inspection object will be described. Since structures of the individual sub-coils 420 constituting the coil unit 410 are common, one sub-coil will be described as a representative.

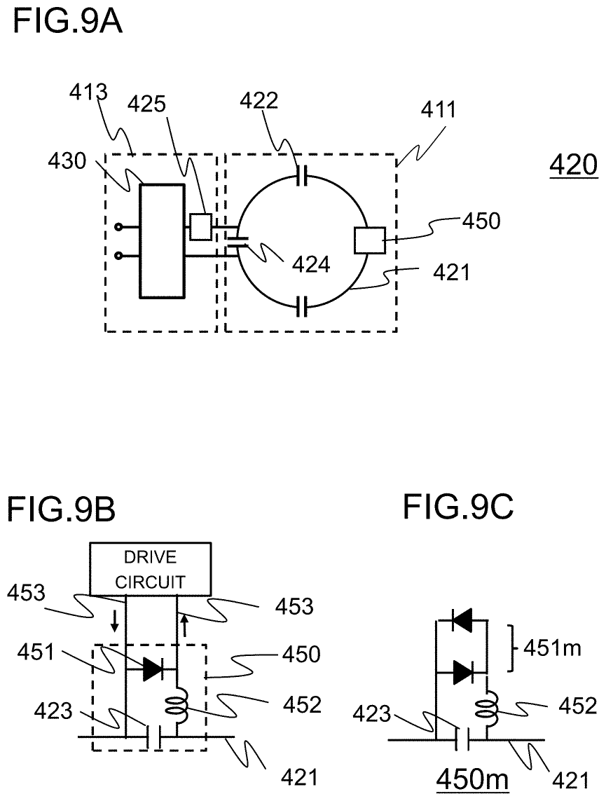

FIG. 9A is a block diagram for explaining a structure of one sub-coil 420 among the plurality of sub-coils constituting the coil unit (array coil) 410 of the present embodiment. The sub-coil 420 is a surface coil having a conductor loop as shown in FIGS. 4A and 4B, and each receives the nuclear magnetic resonance signal. The received signal is sent to a receiver (not shown) of the MRI apparatus.

The sub-coil 420 includes the loop coil portion 411 that receives the nuclear magnetic resonance signal and the power supply substrate 413. The power supply substrate 413 includes the low (input) impedance signal processing circuit 430 and a magnetic coupling adjustment unit 425 that connects the loop coil portion 411 and the low impedance signal processing circuit 430. The magnetic coupling adjustment unit 425 includes at least one of the capacitor and an inductor.

A loop 421 of the loop coil portion 411 is formed of the conductor. The loop coil portion 411 includes a capacitor 424 inserted in series into an inductor component of the loop 421. The inductor component and the capacitor 424 constitute the parallel resonance circuit. The capacitor 424 is referred to as the parallel capacitor 424 in order to distinguish it from other capacitors.

Further, a capacitor 422 that adjusts the resonance frequency and a transmission-reception magnetic coupling prevention circuit 450 (FIG. 2: 145) are inserted in series into the loop 421. The capacitor 422 is referred to as the series capacitor 422 to distinguish it from other capacitors. Here, a case where two series capacitors 422 are provided is illustrated, however, the number of series capacitors 422 only needs to be one or more.

As described above, the sub-coil 420 of the present embodiment includes the magnetic coupling adjustment unit 425 as the circuit element for adjustment, the series capacitor 422 inserted in series into the inductor component of the loop 421, and the parallel capacitor 424 that is inserted in series into the inductor component and has the loop coil portion 410 as the parallel resonance circuit.

One terminal on the loop coil portion 411 side of the low impedance signal processing circuit 430 is connected to one end of the parallel capacitor 424 of the loop coil portion 411 through the magnetic coupling adjustment unit 425. The other terminal on the loop coil portion 411 side of the low impedance signal processing circuit 430 is directly connected to the other end of the parallel capacitor 424 of the loop coil portion 411. The other terminal of the low impedance signal processing circuit 430 that is not on the loop coil portion 411 side is connected to the receiver of the MRI apparatus through a transmission cable.

The transmission-reception magnetic coupling prevention circuit 450 releases magnetic coupling with the transmission coil. A circuit example of the transmission-reception magnetic coupling prevention circuit 450 is shown in FIGS. 9B and 9C. The transmission-reception magnetic coupling prevention circuit 450 shown in FIG. 9B includes a PIN diode 451 and an inductor 452 connected in series, and is connected in parallel to a capacitor 423 inserted into the loop 421. The inductor 452 and the capacitor 423 are adjusted to resonate in parallel at the frequency of the received nuclear magnetic resonance signal.

Further, both ends of the PIN diode 451 are connected with a control signal line 453 connected to the magnetic coupling prevention circuit driving device 180 (FIG. 2) provided on the MRI apparatus side. A choke coil (not shown) is inserted into the control signal line 453 in order to avoid high frequency mixing.

In the transmission-reception magnetic coupling prevention circuit 450, when current flows through the PIN diode 451 forming the parallel resonance circuit by a signal from the control signal line 453, the PIN diode 451 is turned on, and the capacitor 423 of the loop 421 resonates in parallel with the inductor 452 at the frequency of the received nuclear magnetic resonance signal to enter a high impedance state (high resistance). Therefore, a part of the loop coil portion 411 becomes high impedance to be in an off-state at the frequency of the received nuclear magnetic resonance signal, and the sub-coil 420 having the loop coil portion 411 is also in the off-state.

Thus, when the current flows through the PIN diode 451 to be turned on, the magnetic coupling between each sub-coil and the RF transmitter coil 130 (FIG. 2) is released. Accordingly, the magnetic coupling between the array coil (coil unit) 410 having each sub-coil 420 as a coil element and the RF transmitter coil 130 is also released.

The number of transmission-reception magnetic coupling prevention circuits 450 inserted into the sub-coil 420 is not limited to one. Two or more may be inserted into each loop 421. The magnetic coupling can be sufficiently reduced by inserting a plurality of transmission-reception magnetic coupling prevention circuits.

A transmission-reception magnetic coupling prevention circuit 450m shown in FIG. 9C uses a cross diode 451m instead of the PIN diode 451. Thus, when a large signal flows through the conductor constituting the loop 421, the cross diode 451m is turned on, and the capacitor 423 of the loop 421 resonates in parallel with the inductor 452 at the frequency of the received nuclear magnetic resonance signal to enter the high impedance state. In this case, the control signal line is not required.

Next, in the above structure, adjustment for preventing the magnetic coupling between the sub-coils 420 will be described.

Capacitance of the parallel capacitor 424 is adjusted such that the impedance at the resonance frequency of the parallel resonance circuit of the loop coil portion 411 viewed from the low impedance signal processing circuit 430 is equivalent to a characteristic impedance (for example, 50Ω) of the transmission cable to which the low impedance signal processing circuit 430 is connected, when the coil units 410A and 410B are arranged in close contact with the head to be inspected. The parallel capacitor 424 functions to perform impedance conversion. As a distance between the loop coil portion 411 and the subject 20 is smaller, a biological load is larger, and the capacitance of the parallel capacitor 424 becomes smaller in order to adjust impedance of the resonance circuit.

Further, the magnetic coupling adjustment unit 425 (capacitor or the like) is adjusted, so that the resonance circuit including the magnetic coupling adjustment unit 425 and the parallel capacitor 424 resonates in series at the frequency of the received nuclear magnetic resonance signal when viewed from the low impedance signal processing circuit 430. This resonance circuit is the parallel resonance circuit when viewed from both ends of the parallel capacitor 424. In the parallel resonance circuit, since input impedance of the low impedance signal processing circuit 430 is low as described above, the impedance at the both ends of the parallel capacitor 424 at the resonance frequency is increased. Therefore, when viewed from the other sub-coil, a part of the loop coil portion 411 of the sub-coil becomes high impedance and the magnetic coupling between the sub-coils is prevented. That is, since the magnetic coupling is prevented by increasing the impedance at the both ends of the parallel capacitor 424 at the resonance frequency, the impedance is referred to as a block impedance here.

Specifically, when angular frequency corresponding to the nuclear magnetic resonance frequency is co, the capacitance of the parallel capacitor 424 is Cₘ, and the input impedance of the low impedance signal processing circuit 430 is Zᵢₙ, the block impedance is expressed by the following equation.

Z_{block}1/(ω²Cₘ²Zᵢₙ) (Equation 1)

Since the capacitance Cₘ of the parallel capacitor 424 is reduced as the distance between the loop coil portion 411 and the subject 20 is reduced, the block impedance can be increased by arranging the coil body and the subject in close contact with each other. Thus, a magnetic coupling prevention function between the sub-coils can be improved, so that the image quality can be prevented from being reduced.

In an adjustment example described above, when the coil units 410A and 410B are arranged in close contact with the head of the subject, the capacitance of the parallel capacitor 424 is adjusted such that the impedance at the resonance frequency of the parallel resonance circuit of the loop coil portion 411 viewed from the low impedance signal processing circuit 430 is equivalent to the characteristic impedance (for example, 50ω) of the transmission cable to which the low impedance signal processing circuit 430 is connected. However, the capacitance of the parallel capacitor 424 may be smaller than the characteristic impedance. Thus, the block impedance can be increased.

Structural characteristics and electrical characteristics of the radio-frequency coil device according to the first embodiment (particularly, a magnetic coupling prevention effect associated with changes in the shape and positional relationship of the coils) have been described above. However, the embodiment shown in the drawings is merely an example, and various modifications can be made. Hereinafter, several modifications based on the first embodiment will be described.

### <Modification 1>

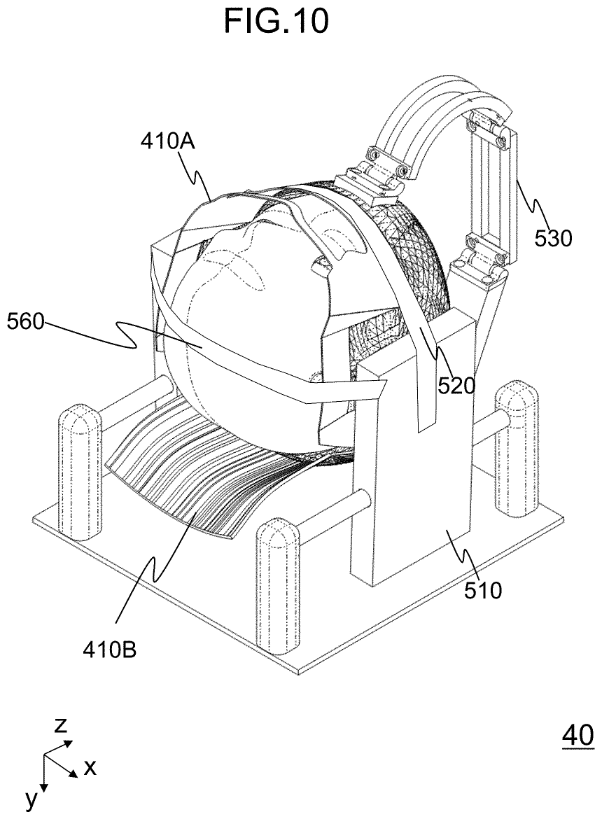

In the first embodiment, the fixture 500 includes only the fixing panel 510 and the fixing belt 520, but is not limited thereto. For example, as shown in FIG. 10, a chin band 560 may be added. The chin band 560 may be fixed to an outside of the panel 510, or may be configured such that the panel 510 and the chin band 560 are respectively provided with hook-and-loop fasteners so that the chin band can be attached and detached. By providing the chin band 560, the body movement in the nodding direction of the subject can be further suppressed.

### <Modification 2>

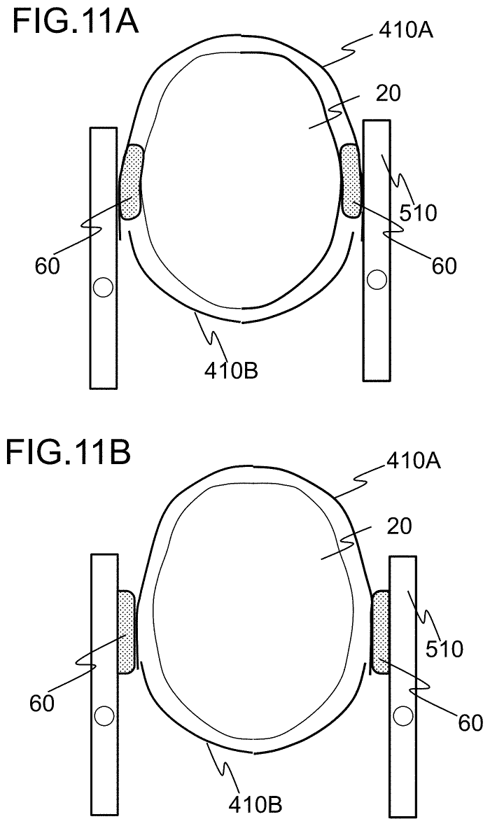

In the panel of the first embodiment, a mechanism (sound insulating member) for suppressing sound may be added to at least one of the coil body 400 and the panel 510. For example, as shown in FIG. 11A, an earmuff mechanism 60 is attached to an inside of the coil unit 410A. Or, as shown in FIG. 11B, a hole is opened in a portion of the coil unit 410A that contacts an ear of the subject 20, and the earmuff mechanism 60 is attached to the panel 510. Thus, noises heard by the subject during MRI examination can be reduced. Further, a work time for additionally attaching earmuffs or earplugs can be reduced.

As described above, a case where the radio-frequency coil device according to the first embodiment and the modifications is applied to the MRI apparatus of the horizontal magnetic field type has been described. However, the radio-frequency coil devices can also be applied to the MRI apparatus of the vertical magnetic field type.

### Second Embodiment

The first embodiment is an embodiment in which the radio-frequency coil device of the present invention is used as the receiver coil of the MRI apparatus. However, in the present embodiment, the radio-frequency coil device is used as the transmitter coil. Also in the present embodiment, it is the same as in the first embodiment that the radio-frequency coil device includes the flexible coil body and the fixture or the fixture and the support unit, and the coil body is deformed by the fixture/the support unit or the distance between the coil units is changed, so that the radio-frequency coil device is fixed to be in close contact with the subject, and the modifications of the fixture can also be applied to the present embodiment.

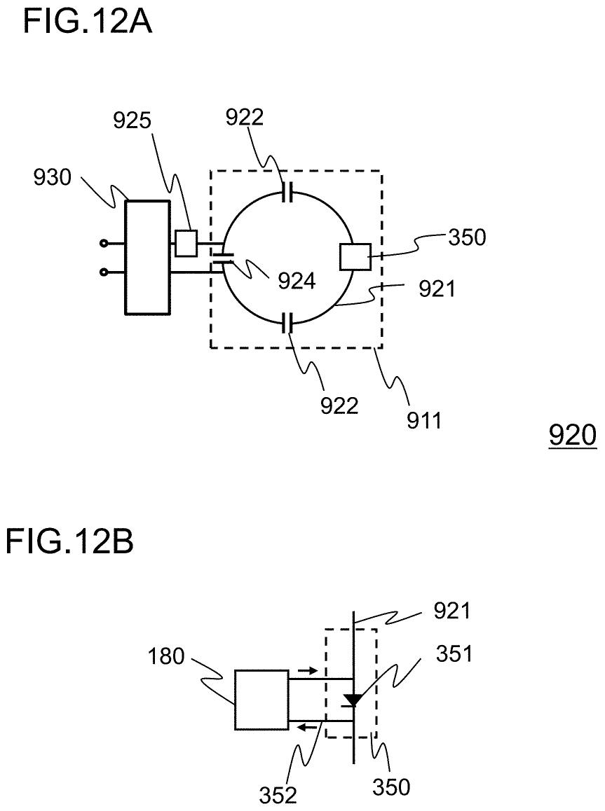

Hereinafter, differences from the first embodiment will be described. FIGS. 12A and 12B are circuit diagrams when a radio-frequency coil device 40B of the present embodiment is used as the transmitter coil. FIGS. 12A and 12B also show one of the sub-coils constituting the coil unit, however, when the coil unit is the array coil including the plurality of sub-coils, the plurality of sub-coils are arranged as shown in FIGS. 4A and 4B, and each sub-coil has the circuit structure of FIGS. 12A and 12B.

As shown in FIG. 12A, each sub-coil 920 of the array coil that operates as the transmitter coil has a loop coil portion 911 and a low output impedance signal processing circuit 930, and includes a magnetic coupling adjustment unit 925 between the loop coil portion 911 and the low output impedance signal processing circuit 930. A series capacitor 922 and a parallel capacitor 924 are inserted in a conductor loop 921 of the loop coil portion 911. This structure is basically the same as the sub-coil 420 (FIG. 9A) of the first embodiment. However, it is not the low input impedance signal processing circuit 430 but the low output impedance signal processing circuit 930 that is connected with the loop coil portion 911 through the magnetic coupling adjustment unit 925. As the low output impedance signal processing circuit 930, a low output impedance RF amplifier (low output impedance signal amplifier) or the like is used.

The sub-coil 920 includes a transmission-reception magnetic coupling prevention circuit 350 (FIG. 2: 135) that prevents magnetic coupling with the receiver coil. Similarly to the transmission-reception magnetic coupling prevention circuit 450 of the receiver coil, the transmission-reception magnetic coupling prevention circuit 350 may be the one shown in FIG. 9B, or a PIN diode 351 connected in series to the conductor loop 921 of the loop coil portion 911 as shown in FIG. 12B. In this case, when the current flows from the control circuit on the MRI apparatus side to the PIN diode 351 by a drive signal 352, the loop coil portion 911 is closed, and the current flows to the loop coil portion 911 by a high-frequency signal supplied through the transmission cable, to generate the high-frequency magnetic field. At this time, when the receiver coil is provided separately from the radio-frequency coil device, the transmission-reception magnetic coupling prevention circuit provided in the receiver coil is operated to turn off the receiver coil. When the radio-frequency coil device is used for both transmission and reception, supply of the high-frequency signal to the loop coil portion 911 and transmission of the drive signal 352 to the transmission-reception magnetic coupling prevention circuit 350 (PIN diode 351) are synchronized, so that transmission-reception switching can be performed.

The structure and adjustment of each circuit element in the array coil (sub-coil 920) are basically the same as those of the array coil (coil unit) 410 of the first embodiment. The adjustment is performed by the series capacitor 922 inserted in series into the loop 921 of the loop coil portion 911, the parallel capacitor 924 inserted in parallel thereinto, and the magnetic coupling adjustment unit 925.

Specifically, the capacitance of the circuit element is adjusted by the parallel capacitor 924 so that the resonance frequency of each sub-coil 920 that is the parallel resonance circuit is the same as the nuclear magnetic resonance frequency of the MRI apparatus. Further, the resonance circuit including the magnetic coupling adjustment unit 925 and the parallel capacitor 924 is adjusted to resonate in series at the frequency of the nuclear magnetic resonance signal when viewed from the low impedance signal processing circuit 930. The resonance circuit is the parallel resonance circuit when viewed from both ends of the parallel capacitor 924, and the capacitance of the parallel capacitor 924 is smaller as an array coil 910 is brought into closer contact with the subject 20. Therefore, the impedance (block impedance) at the both ends of the parallel capacitor 924 is increased by the above equation (1), to prevent the magnetic coupling between the sub-coils.

As described above, the sub-coils 920 constituting the transmitting array coil 910 of the present embodiment resonate at a desired frequency (for example, 64 MHz), so that RF can be efficiently transmitted. At the same time, since the array coil can be disposed in close contact with various head shapes, the sensitivity is improved as in the first embodiment. Therefore, transmission efficiency is improved and power required for imaging can be reduced.

Further, the sub-coils 920 are not coupled to each other and have a sensitivity region different from that of other sub-coils. Therefore, they function as a multi-channel.

As in the first embodiment, the present embodiment can also be applied to both the MRI apparatus provided with a horizontal magnetic field type magnet and the MRI apparatus provided with a vertical magnetic field type magnet.

What is claimed is:

1. A radio-frequency coil device for a magnetic resonance imaging apparatus, comprising:
a flexible coil unit; and
a fixture disposed outside the coil unit with respect to an inspection object, wherein
the fixture includes a fastener that brings the coil unit attached to the inspection object into close contact with the inspection object.

2. The radio-frequency coil device according to claim 1, wherein the coil unit has a plurality of loop coil portions, and a distance between the loop coil portions is variable.

3. The radio-frequency coil device according to claim 1, wherein the fixture includes a pair of panels that are disposed on both sides of the inspection object and have an adjustable distance from each other, and a fixing belt that fastens the pair of panels as the fastener.

4. The radio-frequency coil device according to claim 1, further comprising a support unit that supports the coil unit and fixes the coil unit to the inspection object at a position different from both sides of the inspection object.

5. The radio-frequency coil device according to claim 4, wherein the support unit includes a mechanism for moving the coil unit between an inspection position and a retracted position.

6. The radio-frequency coil device according to claim 1, wherein
the coil unit is provided in plurality, and
the fixture brings the coil units into close contact with the inspection object by changing a distance between the coil units.

7. The radio-frequency coil device according to claim 6, wherein the coil unit includes a first coil unit that covers a frontal side of the inspection object and a second coil unit that covers an occipital side of the inspection object, and is a coil for a head.

8. The radio-frequency coil device according to claim 7, wherein the first coil unit includes a viewing window for the subject.

9. The radio-frequency coil device according to claim 7, wherein at least one of the first coil unit and the fixture is provided with a sound insulating member that blocks sound against the inspection object.

10. The radio-frequency coil device according to claim 1, wherein
the coil unit includes a first coil unit that covers a frontal side of the inspection object, and a second coil unit that covers an occipital side of the inspection object,
a support unit that supports the first coil unit and fixes the first coil unit to the inspection object at a position different from both sides of the inspection object is further provided, and
the support unit has a mechanism for moving the first coil unit between a first inspection position and a second inspection position different from the first inspection position.

11. The radio-frequency coil device according to claim 1, wherein
the coil unit is an array coil including a plurality of sub-coils,
each sub-coil has a loop coil portion, a parallel capacitor that is inserted in series with respect to an inductor component of the loop coil portion and has the loop coil portion as a parallel resonance circuit, and a low impedance signal processing circuit that is connected in parallel with the parallel capacitor and connected with a transmission cable, and
capacitance of the parallel capacitor is adjusted to be less than or equal to the capacitance at which impedance at a resonant frequency of the parallel resonance circuit is equivalent to a characteristic impedance of the transmission cable.

12. The radio-frequency coil device according to claim 1, having a shape that covers a human head, wherein
the coil unit is an array coil including a plurality of sub-coils, and each sub-coil has a loop coil portion and a circuit substrate including an impedance signal processing circuit to which a transmission cable is connected, and
the circuit substrate is disposed concentrated on a parietal side.

13. The radio-frequency coil device according to claim 1, wherein the coil unit is a receiver coil that receives a nuclear magnetic resonance signal of the magnetic resonance imaging apparatus or a transmitter coil for applying a high-frequency magnetic field of the magnetic resonance imaging apparatus.

14. A magnetic resonance imaging apparatus comprising:
a magnet that generates a static magnetic field;
a gradient magnetic field coil that generates a gradient magnetic field in a space of the static magnetic field formed by the magnet;
a transmitter coil that applies a high-frequency magnetic field to a subject placed in the space of the static magnetic field; and
a receiver coil that receives a nuclear magnetic resonance signal generated from the subject, wherein
at least one of the transmitter coil and the receiver coil is a radio-frequency coil device according to claim 1.

15. The magnetic resonance imaging apparatus according to claim 14, wherein the radio-frequency coil device is disposed so that a loop surface of a loop coil portion included in the coil unit is parallel to a direction of the static magnetic field.
